(19) United States
(12) Patent Application Publication
(10) Pub. No.: US 2026/0337143 A1
(43) Pub. Date: Oct. 29, 2026

(54) PROCESS FOR RECOVERING VALUE FROM ACRYLIC HEAVIES VIA ESTERS

(71) Applicant: Rohm and Haas Company, Collegeville, PA (US)

(72) Inventors: Joseph F. Dewilde, King of Prussia, PA (US); Mark A. Silvano, Upper Black Eddy, PA (US); Muhunthan Sathiosatham, Chalfont, PA (US); Lan T.P. Hoang Nguyen, Doylestown, PA (US)

(21) Appl. No.: 19/391,352

(22) Filed: Nov. 17, 2025

Related U.S. Application Data
(60) Provisional application No. 63/728,474, filed on Dec. 5, 2024.

(51) Int. Cl. C07C 57/04; C07C 51/43; C07C 51/487; C07C 67/08; C07C 69/54; C07C 69/593

(52) U.S. Cl. CPC C07C 57/04; C07C 51/43; C07C 51/487; C07C 67/08; C07C 69/54; C07C 69/593

(57) ABSTRACT

A process for recovering acrylic values from Michael addition products comprises reacting, in a liquid phase, a stream comprising acrylic acid and Michael addition products of acrylic acid with a C1 to C8 alkyl alcohol in the presence of an esterification or transesterification catalyst at a temperature ranging from 60 to 120 °C to form an intermediate mixture comprising a C1 to C8 alkyl acrylate and a C1 to C8 alkyl-acryloxy propionate. Excess C1 to C8 alkyl alcohol is removed from the intermediate mixture to form an alkylated heavies stream. The alkylated heavies stream is thermally cracked to produce a mixture of acrylic acid and its corresponding C1 to C8 alkyl ester.

### FIELD OF THE INVENTION

The invention relates to a method for recovering acrylic value from acrylic oligomers.

### BACKGROUND OF THE INVENTION

Acrylic acid and its ester derivatives are used in the manufacture of a broad spectrum of polymer products found across multiple market segments. For example, poly-acrylic acid (PAA) formulations are commonly found in detergent applications, sodium salts of PAA are used as super absorbent polymers (SAP) found in baby diapers and other adult hygiene products. Copolymers of acrylic monomers with methacrylic monomers, styrene, butadiene and other functionalized vinyl monomers are used to manufacture dispersions and resins that are ubiquitous in consumer and industrial products such as architectural, industrial, paper and automotive coatings, performance additives for concrete admixtures and asphalts, textiles, adhesives, sealants and caulks to name just a few applications.

Commercially, acrylic monomer production is dominated by acrylic acid which serves as a platform chemical for a broad range of acrylate derivatives. Acrylic acid may be produced using a variety of different methods. For example, acrylic acid can be made via propylene oxidation, bio-based processes (e.g., using any one of glycerol, 3-hydroxypropionic acid, lactate, lactic acid, etc., as a starting material), the ethylene cyanohydrin process, the acetylene process (i.e., the Reppe process), the β-propiolactone process, acrylonitrile hydrolysis, or coupling carbon dioxide with ethylene or ethane. Industrially, most of the global capacity of acrylic acid is manufactured via the two-stage heterogeneously catalyzed oxidation of propylene over mixed metal oxide catalysts.

The crude acrylic acid (CAA) that is produced by any of the known methods contains Michael addition products, which include dimers, trimers, and higher -mers of acrylic acid. These Michael addition products may reversibly react to form acrylic acid or higher or lower oligomers.

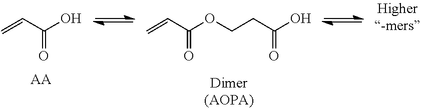

Michael addition products of acrylic acids are also present in waste streams and bottoms streams from acrylic acid purification processes, as well as waste and bottoms streams from ester production processes that use acrylic acid in the formation of the esters, such as, for example, butyl acrylate production.

Dimer crackers are typically used to recover acrylic acid value from the Michael addition products present in these streams.

Attempts have been made to improve acrylic acid recovery in dimer crackers. Some methods utilize strong acids as a catalyst to facilitate recovery of acrylic value.

European Patent EP 1275633 discloses a process for decomposing Michael adducts of acrylic acid by reacting in the presence of an N-oxyl compound to form acrylic acid, acrylic ester, and/or alcohol.

U.S. Pat. No. 7,612,231 discloses a process for recovering acrylic acid from a mixture comprising acrylic acid dimer by carrying out decomposition of the acrylic acid dimer under reduced pressure while recovering acrylic acid by distillation in a process having a retention time at the bottom of the acrylic acid recovering device of 10 to 30 hours.

U.S. Pat. No. 8,308,913 discloses an apparatus for recovering acrylic acid comprising an acrylic acid recovering device having an acrylic acid recovery device having a thin film evaporator integrated with an acrylic acid dimer pyrolysis tank.

U.S. Pat. No. 3,868,410 discloses an esterification process wherein oligomeric byproducts are converted to acrylic acid, an alkanol, or a alkylate of the alkanol by heating the oligomeric byproducts at a temperature of at least 180 °C.

The methods disclosed in the references cited above recover at most only 50 wt % of eligible acrylic acid molar equivalents.

There is a need in the art for improved recovery of acrylic acid from Michael addition products.

### SUMMARY OF THE INVENTION

The invention is directed to a process for recovering acrylic values from Michael addition products comprising:
a) reacting, in a liquid phase, a stream comprising acrylic acid and Michael addition products of acrylic acid with a C1 to C8 alkyl alcohol in the presence of an esterification or transesterification catalyst at a temperature ranging from 60 to 120 °C to form an intermediate mixture comprising a C1 to C8 alkyl acrylate and a C1 to C8 alkyl-acryloxy propionate;
b) removing excess C1 to C8 alkyl alcohol from the intermediate mixture to form an alkylated heavies stream; and
c) thermally cracking the alkylated heavies stream to produce a mixture of acrylic acid and its corresponding C1 to C8 alkyl ester.

### BRIEF DESCRIPTION OF THE DRAWINGS

FIG. 1 is a flow diagram showing the process according to one embodiment of the present invention.

FIG. 2 is a diagram of the chemical reactions associated with the process according to an embodiment of the present invention.

### DETAILED DESCRIPTION OF THE INVENTION

All percentage compositions are weight percentages (wt %), and all temperatures are in °C, unless otherwise indicated. Averages are arithmetic averages unless otherwise indicated. An “average concentration” is the arithmetic average of the concentration entering a region and the concentration exiting the region, where the region is an individual reactor, a reactor system, or a zone within a reactor or reactor system.

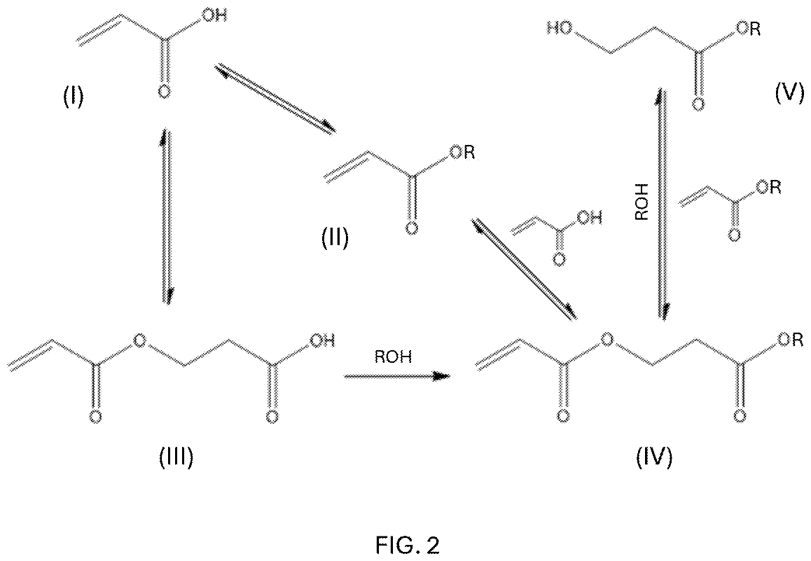

As used herein, the terms “Michael addition products of acrylic acid” and “dimer, trimer, and higher -mers of acrylic acid” refer to oligomers of acrylic acid. The oligomers of acrylic acid can reversibly react to form higher or lower oligomers, as well as acrylic acid. For example, 3-acryloxypropionic acid (AOPA; compound (III) in FIG. 2) may reversibly react to form acrylic acid (compound (I)), as well as the trimer.

The present invention relates to a process for recovering acrylic value from heavy waste byproducts, such as Michael addition products of acrylic acid. As used herein “acrylic value” refers to acrylic acid and esters of acrylic acid that are useful in the production of polymers.

In the inventive process, acrylic value recovery is performed by reacting, in the liquid phase, a stream comprising acrylic acid and Michael addition products of acrylic acid with a C1 to C8 alkyl alcohol (i.e., ROH, where R is a C1 to C8 alkyl group) to form an intermediate mixture comprising a C1 to C8 alkyl acrylate and a C1 to C8 alkyl-acryloxy propionate. The stream comprises acrylic acid, the dimer of acrylic acid (3-(acryloyloxy)propanoic acid or AOPA), acrylic acid trimer, and acrylic acid tetramer. Higher -mers of acrylic acid may also be present. The dimer is the majority component of the Michael addition products. More preferably, the dimer is the majority component in the stream comprising acrylic acid and its Michael addition products. For example, the stream may be a waste stream, the bottoms stream of an acrylic acid production process or a crude or low grade acrylic acid stream.

When reacted with a C1 to C8 alkyl alcohol (shown as ROH in FIG. 2, where R is a C1 to C8 alkyl group), corresponding C1 to C8 alkyl esters of the acrylic acid (i.e., C1 to C8 alkyl acrylates, compound (II)) and its Michael addition products (compound (IV)) are formed, as shown in FIG. 2.

The alkoxy-3-acryloyloxypropanoic acid of formula (compound (IV)) may further react with the C1 to C8 alkyl alcohol to form an alkyl-3-hydroxy propionate (compound (V)) as well as oligomers thereof. To avoid formation of the alkyl-3-hydroxy propionate (compound (V)) and its oligomers, the reaction of the stream comprising acrylic acid and its Michael addition products with a C1 to C8 alkyl alcohol is conducted at a temperature between 60 and 120 °C, preferably from 65 to 100 °C, and more preferably from 70 to 85 °C.

The reaction of the acrylic acid and its Michael addition products with a C1 to C8 alkyl alcohol is catalyzed with an esterification or transesterification catalyst. Preferably, the esterification or transesterification catalyst is a homogeneous acid catalyst. Examples of suitable acid catalysts include hydrochloric acid, sulfuric acid, p-toluenesulfonic acid, methane sulfonic acid, and phosphoric acid. Preferably, the catalyst is sulfuric acid.

The C1 to C8 alkyl alcohol is preferably selected from methanol, ethanol, butanol, and 2-ethyl hexyl alcohol. More preferably, the C1 to C8 alkyl alcohol is butanol.

Optionally, water may be removed from the intermediate mixture before further processing. The water may be removed using phase separation. The aqueous phase may contain impurities, as well as some of the acid catalyst.

Excess C1 to C8 alkyl alcohol from the intermediate mixture to form an alkylated heavies stream. Excess C1 to C8 alkyl alcohol may be removed by evaporation. In one embodiment, the step of removing excess C1 to C8 alkyl alcohol can be performed in the same vessel as the reaction of the C1 to C8 alkyl alcohol with the stream comprising acrylic acid and Michael addition products of acrylic acid. In an alternative embodiment, the removal of excess C1 to C8 alkyl alcohol takes place in a different vessel. For example, the excess C1 to C8 alkyl alcohol may be removed using a flash pot, a distillation column, or other evaporative separation unit.

Any of the C1 to C8 alkyl acrylates formed by the reaction of acrylic acid with the C1 to C8 alkyl alcohol may also be removed with the excess C1 to C8 alkyl alcohol.

Preferably, removal of the excess C1 to C8 alkyl alcohol is performed at a temperature less than 100 °C, more preferably less than 90 °C, and even more preferably less than 80 °C.

Preferably, removal of the excess C1 to C8 alkyl alcohol is performed under vacuum (i.e., less than atmospheric pressure). For example, the excess C1 to C8 alkyl alcohol may be removed at a pressure of 50 mmHg.

Removal of the excess C1 to C8 alkyl alcohol results in an alkylated heavies stream.

The removed excess C1 to C8 alkyl alcohol may be condensed and optionally recycled to the first step of the process.

The alkylated heavies stream is thermally cracked to form a mixture of acrylic acid and its corresponding C1 to C8 alkyl ester (i.e., C1 to C8 alkyl acrylate). The alkylated heavies stream is preferably cracked at an elevated temperature of at least 180 °C, more preferably at least 190 °C, and even more preferably at least 200 °C.

In one embodiment, the alkylated heavies stream is thermally cracked by recirculating the alkylated heavies stream between a pressurized liquid phase reactor and an evacuated flash pot, where the pressurized liquid phase reactor is operated at a higher temperature than the evacuated flash pot. For example, the pressurized liquid phase reactor may be operated at a temperature greater than 180 °C and the evacuated flash pot may be operated at a temperature less than 140 °C. It is believed that the combination of the pressurized liquid phase reactor and the evacuated flash pot aids cracking of the heavy esters by removing the acrylic acid and C1 to C8 alkyl acrylates formed in the pressurized liquid phase reactor to drive the reversible reaction in the direction of additional cracking of the heavy esters.

The acrylic acid and C1 to C8 alkyl acrylates produced may be condensed and collected.

Preferably, a polymerization inhibitor is added at any point in the process or already present in the feed stream fed to the process by virtue of being added during a previous distillation/separation step. For example, a polymerization inhibitor may be added with the C1 to C8 alkyl alcohol or during the thermal cracking step. The polymerization inhibitor may be selected from any known polymerization inhibitor. Examples of preferred inhibitors include soluble manganese ions, soluble copper ions, hydroquinone (HQ), monomethyl ether hydroquinone (MEHQ), phenothiazine (PTZ), 2,2,6,6-tetramethyl-1-piperidinyloxy (TEMPO) and related compounds such as 4-hydroxy TEMPO. Combinations of inhibitors can be employed, such as, for example, a mixture of a source of soluble manganese ions, such as manganese acetate, and 4-hydroxy TEMPO. The inhibitor is employed in an amount sufficient to prevent or reduce the polymerization of acrylic acid, as is well known to those skilled in the art.

The present invention surprisingly enables the recovery of a significant amount of the Michael addition products of acrylic acid from the feed. For example, when butanol is used as the C1 to C8 alkyl alcohol, 90 wt % or more of the Michael addition products of acrylic acid in the acrylic heavies may be recovered as acrylic acid or butyl acrylate. This represents a significant improvement over conventional dimer cracking methods.

Preferably, the stream comprising acrylic acid and Michael addition products of acrylic acid used in the present invention comprises acrylic acid generated in any known acrylic acid production process. For example, acrylic acid can be made via propylene oxidation, a bio-based process (e.g., using any one of glycerol, 3-hydroxypropionic acid, lactate, lactic acid, etc., as a starting material), an ethylene cyanohydrin process, an acetylene process (i.e., the Reppe process), a β-propiolactone process, an acrylonitrile hydrolysis process, or a process performed by coupling carbon dioxide with ethylene or ethane. The reaction mixture produced during the acrylic acid production process may be processed in one or more unit operations. These operations may include, for example, cooling processes, quenching processes, separations, purifications, etc., or a downstream reaction such as an ester production process.

Preferably, the stream used in the inventive process comprises a low grade acrylic acid stream, a waste stream, or a bottoms stream from a purification process that draws off a product stream that is substantially comprised of acrylic acid, such as, for example, a product stream comprising at least 90 wt % acrylic acid. The liquid stream also, or alternatively, comprises a waste stream or a bottoms stream from an ester production process, preferably an alkyl acrylate production process. The low grade acrylic acid stream, waste stream, or bottoms stream comprises Micheal addition products of acrylic acid and at least some amount of acrylic acid from the acrylic acid production process and is reacted with the C1 to C8 alkyl alcohol to recover acrylic values.

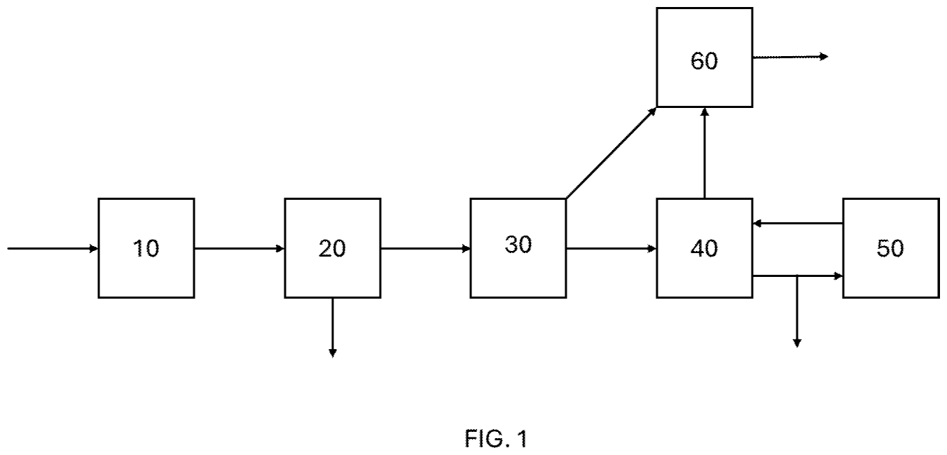

One embodiment of an apparatus used to perform the inventive process is shown in FIG. 1. A stream comprising acrylic acid and Michael addition products of acrylic acid is fed to a reactor 10, along with a C1 to C8 alkyl alcohol and an acid catalyst. The intermediate stream exiting reactor 10 is fed to a phase separator 20 to remove the aqueous phase. The remaining intermediate stream is fed to a flash pot 30 to remove excess C1 to C8 alkyl alcohol. Alternatively, the excess C1 to C8 alkyl alcohol may be removed in reactor 10 as a vapor stream. The vapor from the flash pot 30 is sent to a condenser 60. The alkylated heavies are sent to an evacuated flash pot 40, which is in recirculation with pressurized liquid phase reactor 50. Heavies waste can be removed from the recirculating alkylated heavies stream. The vapor phase containing the recovered acrylic acid and C1 to C8 alkyl acrylate is removed from the evacuated flash pot 40. As shown in FIG. 1, a common condenser 60 is used to condense both the recovered C1 to C8 alkyl alcohol, acrylic acid and C1 to C8 alkyl acrylates. However, separate condensers can also be used.

### Example

Step 1—Liquid phase reaction at lower temperature: a 500 ml glass jacketed flask was charged with 380 g of mixed reactor material comprising 50 wt % acrylate heavies feed, 45 wt % butanol, and 5 wt % sulfuric acid. The mixture was stirred with an impeller and heated to 75 °C under atmospheric pressure. Once at temperature, the same ratio of feed material was fed and reactor fluid was drained to maintain a residence time of 150 min. This mixture was fed until 6000 g of total feed was fed (3000 g of acrylate heavies feed).
Step 2—Removal of aqueous layer formed in Step 1 (optional): The collected bottoms were piecewise placed in a standard glass separatory funnel. Using a flashlight to distinguish the phase interface, the bottom aqueous layer was removed and separated. Each layer was then analyzed for organic composition via gas chromatography (GC) and sulfuric content via titration.
Step 3—Light Compound Flash: The organic portion of Step 2 was fed to a glass three-neck flask acting as a distillation pot at 67 °C and 50 mmHg absolute. An additional stream of water in 0.05:1 mass ratio with the organic feed was also fed to the unit. The liquid level was adjusted such that a feed-based residence time of 15 minutes was maintained. The distillate was gravity separated into organic and aqueous phases after condensing in a water-cooled condenser. As with step 2, both distillate phases as well as the reactor bottoms were analyzed via GC and titration to determine composition.
Step 4—Recirculating High Temperature Reaction and Flash Pot: The setup in this step consists of a glass three-neck flask used for the flashing of product material (similar to step 3) and a liquid phase tube packed with glass spheres heated by an external oil heating bath loop. Fluid was set up to rapidly recirculate between the flash vessel and the packed bed to maintain a well-mixed behavior for the two vessels (recirculation time ~15 min). The void volume of this packed bed was such that it was 34% of the liquid level in the flash pot. The flash pot was kept at 80 °C and 50 mmHg absolute. The liquid phase reactor was kept at 200 °C and >300 psi to maintain fluid in liquid phase. The outlet of the liquid phase reactor was fed through a water cooled heat exchanger to cool the fluid prior to depressurization over a back pressure regulator near the recirculation entrance. A small bleed was maintained to control fluid level at the bottom of the flash pot—representing the bottoms of the combined process. The bottoms from Step 3 was fed with water (0.1:1 mass ratio water:Step3 bottoms) to maintain a 160 min feed-based residence time in the combination of the two vessels. As with Step 3, the distillate from the flash vessel portion was condensed and phase separated. Following the other steps, GC and titration measured the compositions of the two distillate layers and the bottoms.

After performing Step 1 and 2 as described above with a total cumulative feed of 3000 g acrylate heavies feed, 2700 g of butanol, and 300 g of sulfuric acid. Approximately 5600 g of organic layer and 130 g of aqueous layer (96% mass balance across these two steps) were collected. Then after taking 5000 g of this organic layer and a cumulative water feed of 270 g of water in Step 3, 1770 g of organic distillate, 630 g of aqueous distillate and 3140 g of process bottoms (mass balance around 105%) were recovered. Finally, 340 g of the bottoms from Step 3 and with 35 g of additional cumulative water feed of 35 g were treated through the process described in Step 4. As a result, 300 g of organic distillate, 57 g of aqueous distillate, and produced only 31 g of bottoms (total mass balance of ~102%) were recovered.

TABLE 1
All Compositions in wt Fraction

| | | | | | | | | | | |
|---|---|---|---|---|---|---|---|---|---|---|
| | | | | | | Acrylic | | | | |
| | | Higher | | Butyl-3- | Butyl-3- | Dimer | | | | |
| Acrylic | Acrylate | Acrylate | Butyl | butoxy | hydroxy | Butyl | n- | Dibutyl | | Sulfuric |
| Acid | Dimer | Heavies | Acrylate | Propionate | Propionate | Ester | Butanol | Ether | Water | Acid |

Steps 1 and 2 Organic Layer

| | | | | | | | | | | |
|---|---|---|---|---|---|---|---|---|---|---|
| 0.057 | 0.020 | 0.000 | 0.286 | 0.004 | 0.061 | 0.317 | 0.180 | 0.000 | 0.038 | 0.034 |

Steps 1 and 2 Aqueous Layer

| | | | | | | | | | | |
|---|---|---|---|---|---|---|---|---|---|---|
| 0.031 | 0.000 | 0.000 | 0.001 | 0.000 | 0.001 | 0.000 | 0.022 | 0.000 | Balance | 0.342 |

Step 3 Organic Distillate

| | | | | | | | | | | |
|---|---|---|---|---|---|---|---|---|---|---|
| 0.047 | 0.000 | 0.000 | 0.718 | 0.000 | 0.001 | 0.000 | 0.257 | 0.000 | 0.027 | 0.000 |

Step 3 Aqueous Distillate

| | | | | | | | | | | |
|---|---|---|---|---|---|---|---|---|---|---|
| 0.012 | 0.000 | 0.000 | 0.001 | 0.000 | 0.000 | 0.000 | 0.033 | 0.000 | Balance | 0.000 |

Step 3 Bottoms

| | | | | | | | | | | |
|---|---|---|---|---|---|---|---|---|---|---|
| 0.034 | 0.019 | 0.000 | 0.197 | 0.010 | 0.093 | 0.449 | 0.091 | 0.000 | 0.013 | 0.101 |

Step 4 Organic Distillate

| | | | | | | | | | | |
|---|---|---|---|---|---|---|---|---|---|---|
| 0.083 | 0.000 | 0.000 | 0.754 | 0.005 | 0.003 | 0.004 | 0.063 | 0.037 | 0.017 | 0.000 |

Step 4 Aqueous Distillate

| | | | | | | | | | | |
|---|---|---|---|---|---|---|---|---|---|---|
| 0.024 | 0.000 | 0.000 | 0.002 | 0.000 | 0.000 | 0.000 | 0.020 | 0.000 | Balance | 0.000 |

Step 4 Bottoms

| | | | | | | | | | | |
|---|---|---|---|---|---|---|---|---|---|---|
| 0.119 | 0.004 | Balance | 0.092 | 0.032 | 0.041 | 0.068 | 0.184 | 0.002 | 0.023 | 0.050 |
| | | Assumed | | | | | | | | |

The compositions (wt fraction) of each of these streams is included in the table above. Using these and the recovered masses, we were able to define what fraction of “C3” portions are present in the remaining bottoms for each steps: defined as the number of moles of C3 fragments in the bottoms (for example 2× the number of moles of acrylic dimer) compared to the sum total number of moles of C3 units fed to the Step described. For the bottoms of step 4, because we did not have complete analytical closure, we made the conservative assumption of assuming all unaccounted composition (40 wt %) was lost C3, making the reported yields the lower end interpretation of the data. Doing so, we calculated the following:
Fractional C3 loss present in the aqueous stream of Steps 1 and 2 ~0.0015
Fraction of C3 fed to Step 3 present in the bottoms of Step 3 ~0.61
Fraction of C3 fed to Step 4 in the bottoms of Step 4 ~0.11
   To represent the total lost C3 we multiplied the last two of these numbers (as the bottoms of Step 3 was fed to Step 4) and add the first (as the aqueous stream was not fed downstream). There was found an overall fractional loss of ~0.067, or in other words, an acrylate value recovery efficiency of >93%.

1. A process for recovering acrylic values from Michael addition products comprising:
a) reacting, in a liquid phase, a stream comprising acrylic acid and Michael addition products of acrylic acid with a C1 to C8 alkyl alcohol in the presence of an esterification or transesterification catalyst at a temperature ranging from 60 to 120 °C to form an intermediate mixture comprising a C1 to C8 alkyl acrylate and a C1 to C8 alkyl-acryloxy propionate;
b) removing excess C1 to C8 alkyl alcohol from the intermediate mixture to form an alkylated heavies stream; and
c) thermally cracking the alkylated heavies stream to produce a mixture of acrylic acid and its corresponding C1 to C8 alkyl ester.

2. The process of claim 1, wherein the C1 to C8 alkyl alcohol is selected form the group consisting of methanol, ethanol, butanol, and 2-ethyl hexyl alcohol.

3. The process of claim 2, wherein the C1 to C8 alkyl alcohol is butanol.

4. The process of claim 1, wherein step a) is performed at a temperature ranging from 65 to 100 °C.

5. The process of claim 1, wherein removing excess C1 to C8 alkyl alcohol from the intermediate mixture is performed at temperature less than 100 °C.

6. The process of claim 5, wherein removing excess C1 to C8 alkyl alcohol from the intermediate mixture is performed at temperature less than 90 °C.

7. The process of claim 1, wherein removing excess C1 to C8 alkyl alcohol from the intermediate mixture is performed in a flash pot, distillation column, or other evaporative separation unit.

8. The process of claim 1, wherein removing excess C1 to C8 alkyl alcohol from the intermediate mixture is performed under vacuum.

9. The process of claim 1, wherein thermally cracking the alkylated heavies stream comprises recirculating the alkylated heavies stream between a pressurized liquid phase reactor and an evacuated flash pot, wherein the pressurized liquid phase reactor is operated at a higher temperature than the evacuated flash pot.

10. The process of claim 9, wherein the pressurized liquid phase reactor is operated at a temperature greater than 180 °C and the evacuated flash pot is operated at a temperature less than 140 °C.

11. The process of claim 10, wherein the pressurized liquid phase reactor is operated at a temperature greater than 190 °C.

12. The process of claim 1, further comprising a phase separation step to remove water from the intermediate mixture prior to removing the excess C1 to C8 alkyl alcohol.

13. The process of claim 1, wherein the acrylic acid in the stream comprising acrylic acid is produced by a method selected from the group consisting of propylene oxidation-based process, a bio-based process, an ethylene cyanohydrin-based process, an acetylene-based process, a β-propiolactone-based process, an acrylonitrile hydrolysis-based process, or a process performed by coupling carbon dioxide with ethylene or ethane.
